(19) United States
(12) Patent Application Publication
(10) Pub. No.: US 2002/0012702 A1
(43) Pub. Date: Jan. 31, 2002

(54) Controlled release anti-inflammatory oral formulation and a process for the preparation thereof

(75) Inventors: Biyani Milind Kesharlal, Mumbai (IN); Jathar Shripad Rhushikesh, Mumbai (IN); Acharya Manutosh Manohar, Aurangabad (IN)

Correspondence Address: Richard P. Berg, Esq., c/o LADAS & PARRY, 5670 Wilshire Boulevard, Suite 2100, Los Angeles, CA, 90036-5679, US

(73) Assignee: Ajanta Pharma, Ltd.

(21) Appl. No.: 09/884,285

(22) Filed: Jun. 19, 2001

(30) Foreign Application Priority Data
Jun. 20, 2000 (IN) 561/MUM/2000

(51) Int. Cl. A61K 9/14; A61K 9/20

(52) U.S. Cl. 424/488

(57) ABSTRACT

A controlled release anti-inflammatory nimesulide oral formulation. The formulation comprises 45-65% by weight of nimesulide and 10-30% by weight of hydrophilic polymer as retardant with pharmaceutically acceptable auxiliary agents and/or excipients. The nimesulide is partially or fully dispersed in the hydrophilic polymer. The process for the preparation of the formulation comprises mixing the nimesulide with the hydrophilic polymer and pharmaceutically acceptable auxiliary agents and/or excipients.

### FIELD OF THE INVENTION

This invention relates to a controlled release anti-inflammatory nimesulide oral formulation and a process for the preparation thereof.

### PRIOR ART

Nimesulide (4-Nitro-2-phenoxymethane sulfonanilide) is known to be a potent non-steroidal anti-inflammatory drug (NSAID) useful in the treatment of painful inflammatory conditions. It is also reported to have analgesic and antipyretic activity. Pharmacologically, it is a selective cyclo-oxygenase-2 enzyme inhibitor (cox-2). Hence it is less likely to cause gastrointestinal tract side effects. Its metabolites also exhibit anti-inflammatory and analgesic action. Nimesulide has a favourable therapeutic index, minimal acute gastrointestinal toxicity and exhibits good general tolerability. Its pharmacokinetic profile makes it safe even in patients with moderate renal failure. Nimesulide is a hydrophobic drug and is insoluble in water (aqueous solubility is 10 μg/ml at room temperature). Since it is a weak acidic drug its aqueous solubility in acidic medium, for example in the pH of gastric juice particularly, is poor. Hence, orally administered nimesulide is likely to be absorbed only in the lower pat of the gastrointestinal tract. Nimesulide is a time tested drug for the past two decades and has proven safety and efficacy. It is rated to be one of the best amongst the available NSAIDS.

U.S. Pat. No. 5,744,165 discloses inclusion complexes of nimesulide alkali and alkaline earth salts with cyclodextrin or derivative thereof and compositions comprising the same. Injectable nimesulide compositions with parenteral absorption enhancing base such as dimethyl acetamide, benzyl benzoate or ethyl oleate for intramuscular administration are known (U.S. Pat. No. 5,688,829). U.S. Pat. No. 5,756,546 describes water-soluble salt of nimesulide with L-lysine, L-arginine and/or cyclodextrin. Nimesulide for external use dispersed in base component such as cream base like carboxyvinyl polymer, oily substance like diisopropyl myristate and a non-ionic surface active agent is known (U.S. Pat. No. 5,837,735). Nimesulide composition with a percutaneous absorption enhancing vehicle/base such as C₁₂₋₂₄ mono or poly unsaturated fatty acids/alcohols, thickening agent and surfactant in water for topical/transdermal use is known (U.S. Pat. No. 5,716,609). PCT Publication No WO 98/47501 describes nimesulide preparations in a hydroalcoholic solution and a mixture of traditional vehicles and excipients for local use and application to the oral and rhinopharyngeal cavity for the treatment of inflammation of oral and rhinopharyngeal mucosa. U.S. Pat. No. 6,027,747 describes a process for the production of a solid dispersion comprising at least one therapeutic agent in a hydrophilic carrier having enhanced solubility in an aqueous media, by dissolving at least one therapeutic agent in a volatile organic solvent containing a hydrophilic polymer and evaporating the solvent to dryness to form a coprecipitate of the therapeutic agent and the hydrophilic polymer. U.S. Pat. No. 6,048,541 discloses compositions useful for making tablets which can be formed using conventional tabletting machines and which disintegrate rapidly in mouth with optional chewing.

Nimesulide has a short half life of 3-4 hours and therefore requires twice a day administration. The above formulations of nimesulide are of the immediate release type and are recommended in doses of 100-200 mg twice daily. Multiple dosing leads to peas and troughs in nimesulide blood levels. The peek levels of nimesulide may result in undesirable effects or toxicity, while trough levels of nimesulide may be subtherapeutic and may not be very effective. Since nimesulide has been recommended as therapeutic in chronic conditions such as rheumatoid and non-rheumatoid disorders, the drug is required to be present in the blood for long periods of time. It is reported that when the drug is administered in the presence of food, nimesulide plasma concentrations were 2 to 4 fold higher than under fasting conditions suggesting that the drug is better absorbed after meals [“The Pharmacokinetic profile in healthy volunteers”, Drugs 46 (Suppl 1), 1993, p 66, A Bernareggi, published by Adis International]. Therefore if a tablet of the immediate release type is administered after meal at bed time by an arthritic patient, it results in peak effect in the night itself with practically no effect the next morning. Therefore such dosing does not prove useful in reducing early morning stiffness ie immobilization of joints, usually observed in arthritic patients. Further it would necessitate use of another tablet next morning to reduce early morning stiffness. The conventional dosage pattern has poor patient compliance due to such multiple dosage therapy.

### OBJECTS OF THE INVENTION

It is an object of the present invention to provide a controlled release anti-inflammatory nimesulide oral formulation, which is a once-a-day formulation having better patient compliance.

Another object of the invention is to provide a controlled release anti-inflammatory nimesulide oral formulation, which provides for relatively constant and desired nimesulide plasma levels to be therapeutically effective and minimises systemic related side effects.

Another object of the invention is to provide a controlled release anti-inflammatory nimesulide oral formulation, which provides for relatively constant nimesulide plasma levels for a long period of time.

Another object of the invention is to provide a controlled release anti-inflammatory nimesulide oral formulation, which is effective in arthritic conditions, specially in minimising early morning stiffness.

Another object of the invention is to provide a process for the preparation of a controlled release anti-inflammatory nimesulide oral formulation which is a once-a-day formulation having better patient compliance.

Another object of the invention is to provide a process for the preparation of a controlled release anti-inflammatory nimesulide oral formulation which provides for relatively constant and desired nimesulide plasma levels to be therapeutically effective and minuses systemic related side effects.

Another object of the invention is to provide a process for the preparation of a controlled release anti-inflammatory nimesulide oral formulation which provides for relatively constant nimesulide plasma levels for a long period of time.

Another object of the invention is to provide a process for the preparation of a controlled release anti-inflammatory nimesulide oral formulation which is effective in arthritic conditions, specially in minimising early morning stiffness.

### DETAILED DESCRIPTION OF THE INVENTION

According to the invention there is provided a controlled release anti-inflammatory nimesulide oral formulation comprising 45-65% by weight of nimesulide and 10-30% by weight of hydrophilic polymer as retardant with pharmaceutically acceptable auxiliary agents and/or excipients; wherein the nimesulide is partially or fully dispersed in the hydrophilic polymer.

According to the invention there is also provided a process for the preparation of a controlled release anti-inflammatory nimesulide oral formulation comprising mixing 45-65% by weight of nimesulide with 10-30% by weight of hydrophilic polymer as retardant and pharmaceutically acceptable auxiliary agents and/or excipients; wherein the nimesulide is partially or filly dispersed in the hydrophilic polymer.

Preferably nimesulide is in 50-55% by weight in the controlled release formulation of the invention.

According to the invention the nimesulide is dispersed partially or filly in the hydrophilic polymer to form a matrix. In physiological fluid the matrix swells and the nimesulide is released therefrom by diffusion. The hydrophilic polymer used as retardant controls the release of the nimesulide from the polymer matrix. When partially dispersed, that portion of nimesulide dispersed in the hydrophilic polymer matrix is released in a controlled manner whereas the remaining portion of nimesulide not dispersed in the matrix is immediately released in the physiological fluid following administration of the formulation. These formulations are of the partially controlled release type (partially immediate release type). In such ply controlled release formulations as per the invention, 25-65% by weight of the total nimesulide content in the formulation of the invention is used for dispersion in the hydrophilic polymer. When the entire nimesulide is dispersed in the hydrophilic polymer matrix, then a fully controlled release formulation is obtained.

The hydrophilic polymer may be hydroxy propyl methyl cellulose (HPMC), ethyl cellulose, cetostearyl alcohol or the like or mixtures thereof. Preferably HPMC or ethyl cellulose is used.

Preferably the hydrophilic polymer is in 15-20% by weight.

The pharmaceutically acceptable auxiliary agents may be diluent, binder, disintegrant and/or mixtures thereof.

Diluent may be microcrystalline cellulose (MCC), mannitol lactose or the like which provide channeling effect and solubilise the polymer matrix and may be in 2-25%, preferably 5-20% by weight. Preferably diluent used is MCC and/or mannitol.

Binder may be polyvinyl pyrrolidone (PVP), starch, ethyl cellulose, HPMC or the like, preferably PVP and/or starch. Binder may be present in 1-10% by weight, preferably 2-5% by weight.

Disintegrant may be sodium starch glycolate (SSG), cross carmellose sodium, cross povidone, starch or the like, preferably starch and/or SSG. The disintegrant may be in 1-5% preferably 2-3% by weight.

Excipients may be compounds such as dicalcium phosphate dihydrate, sodium citrate, sodium bicarbonate, sodium carboxy methyl cellulose, methyl cellulose or the like or mixtures thereof which cause swelling of the formulations such as tablets resulting in buoyancy of the tablets. These excipients are useful in the preparation of floating tablets which maintain the tablet/drug for longer time at the site of absorption, thereby giving an extended release profile. Excipients may also be lubricants such as talc, colloidal silicone dioxide, magnesium stearate or the like or mixtures thereof in 0.25-4%, preferably 0.5-20% by weight.

The controlled release formulations of the invention may be in the form of tablets, floating tablets or bilayered tablets which maybe coated.

The controlled release formulation of the invention may comprise 50 to 800 mg of nimesulide, preferably 100-600 mg of nimesulide.

According to the invention the release of nimesulide is controlled by the hydrophilic polymeric retardant because of which relatively constant and therapeutically effective or desired nimesulide plasma levels are achieved for extended period of time. Therefore multiple dosage as recommended with conventional dosage is eliminated and the formulation of the invention can be conveniently used as a once-a-day formulation with better patient compliance. The chances of peak and trough nimesulide plasma levels are reduced thereby minimising side effects observed with conventional dosing. Since the drug is made available in the blood for long period of time, the controlled release formulation of the invention has been found to be suitable specially to treat arthritic conditions. The controlled release product of the invention is recommended for use after meal at bedtime. This dosage results in peak levels in the morning, which reduce early morning stiffness usually observed in arthritic patients. The controlled action with the effectiveness of nimesulide has been observed for ˜16 hours.

The following experimental examples are illustrative of the invention but not limitative of the scope thereof.

### EXAMPLE 1

A mix of nimesulide (200 mg), MCC (60 mg), SSG (20 mg) and mannitol (20 mg) sifted through 30 mesh U.S. screen was granulated using PVP (20 mg) dissolved in isopropyl alcohol (50 ml) to get a coherent mass. The wet mix was sifted through 8 mesh U.S. screen and air dried to remove isopropyl alcohol and further dried at 45° C. for 1 hour. The dried granules were sifted through 20 mesh U.S. screen. HPMC (K-4M, 40 mg) and HPMC (K-15M, 24 mg) sifted through 40 mesh U.S. screen was blended with the dried granules and the blend was lubricated with talc (8 mg), magnesium stearate (4 mg) and colloidal silicone dioxide (4 mg) and compressed to tablets.

### EXAMPLE 2

Controlled release tablets comprising 500 mg of nimesulide were prepared as per the procedure of Example 1, but using 2.5 times w/w of each of the ingredients mentioned in Example 1.

### EXAMPLE 3

Controlled release tablets comprising 600 mg of nimesulide were prepared as per the procedure of Example 1, but using 3 times w/w of each of the ingredients mentioned in Example 1.

### EXAMPLE 4

Controlled release tablets comprising 100 mg of nimesulide were prepared as per the procedure of Example 1, but using 0.5 times w/w of each of the ingredients mentioned in Example 1.

### EXAMPLE 5

a) To a mix of nimesulide (100 mg), SSG (7 mg) and MCC (26 mg) sifted through mesh size 20, starch paste was added and the mix was granulated. The granules were sifted through 8 mesh size, dried and further sifted through 20 mesh size. The died granules were mixed with talc (5 mg), magnesium stearate (1.5 mg) and aerosil (2.5 mg) for lubrication. This provided the immediate release fraction.

b) A mix of nimesulide (100 mg) and MCC (57 mg) sifted through 20 mesh size was granulated using PVP (K-30, 6 mg) dissolved in isopropyl alcohol (50 ml). The wet mix was sifted through 8 mesh size, dried and further sifted through 20 mesh size. With these granules was blended a mixture of HPMC (E-10 M, 20 mg), HPMC (K4 MCR, 25 mg) and HPMC (K-100 M, 30 mg). This blend was then lubricated using talc (7 mg), magnesium stearate (2.5 mg) and aerosil (2.5 mg). This provided the controlled release fraction.

c) Granules of steps (a) and (b) were filled in two different hoppers and compressed together using double rotary compression machine to get a bilayered tablet.

### EXAMPLE 6

Controlled release bilayered tablets comprising 300 mg of nimesulide were prepared as per the process of Example 5 having 100 mg of nimesulide as the instant release fraction and 200 mg as the sustained release fraction. The quantities of the ingredients in the formulation were as per Example 5.

### EXAMPLE 7

Controlled release bilayered tablets comprising 400 mg of nimesulide were prepared as per the process of Example 5 having 200 mg of nimesulide as the instant release fraction and 200 mg as the sustained release fraction. The quantities of the ingredients in the formulation were as per Example 5.

### EXAMPLE 8

Controlled release nimesulide tablets comprising 400 mg of nimesulide were prepared as per the process of Example 5 having 100 mg of nimesulide as the instant release fraction and 300 mg as the sustained release fraction. The quantities of the ingredients in the formulation were as per Example 5.

### EXAMPLE 9

Controlled release bilayered tablets comprising 500 mg of nimesulide were prepared as per the process of Example 5 having 200 mg of nimesulide as the instant release fraction and 300 mg as the sustained release fraction. The quantities of the ingredients in the formulation were as per Example 5.

### EXAMPLE 10

Controlled release bilayered tablets comprising 600 mg of nimesulide were prepared as per the process of Example 5 having 300 mg of nimesulide as the instant release fraction and 300 mg as the sustained release fraction. The quantities of the ingredients in the formulation were as per Example 5.

### EXAMPLE 11

Controlled release nimesulide tablets comprising 600 mg of nimesulide were prepared as per the process of Example 5 having 200 mg of nimesulide as the instant release fraction and 400 mg as the sustained release fraction. The quantities of the ingredients in the formulation were as per Example 5.

### EXAMPLE 12

a) A mix of nimesulide (50 mg), SSG (20 mg) and MCC (20 mg) was granulated using PVP (K-30, 2 mg).

b) A mix of nimesulide (150 mg), MCC (40 mg) and mannitol (20 mg) was granulated using PVP (K-30, 15 mg).

c) Granules of steps (a) and (b) were sifted through 20 mesh U.S. screen and mixed together. To the above granule mix was blended HPMC (K4M, 40 mg) and HPMC (K-15M, 24 mg) and the blend was lubricated using talc (8 mg), magnesium stearate (4 mg) and colloidal silicon dioxide (4 mg) and compressed using punches to get bilayered tablets.

### EXAMPLE 13

A mix of nimesulide (200 mg), MCC (70 mg), SSG (10 mg) and mannitol (10 mg) sifted through 30 mesh U.S. screen was granulated using PVP (15 mg) dissolved in isopropyl alcohol to get a coherent mass. The wet mix was sifted through 6 mesh U.S. screen and air dried to remove isopropyl alcohol and fisher dried at 45° C. for 1 hour. The dried granules were sifted through 20 mesh U.S. screen. HPMC (K-4M, 25 mg) and HPMC (K-15M, 50 mg) sifted through 40 mesh U.S. screen were blended with the dried granules. The blend previously sifted through 60 mesh U.S. screen was lubricated with talc (10 mg), magnesium stearate (5 mg) and colloidal silicon dioxide (5 mg) and compressed to tablets.

### EXAMPLE 14

Controlled release tablets comprising 300 mg of nimesulide were prepared as per the procedure of Example 13, but using 300 mg of nimesulide.

### EXAMPLE 15

Controlled release tablets comprising 400 mg of nimesulide were prepared as per the procedure of Example 13, but using 400 mg of nimesulide.

### EXAMPLE 16

Controlled release tablets comprising 200 mg of nimesulide were prepared as per the procedure of Example 13 but the tablets were film coated.

### EXAMPLE 17

Controlled release tablets comprising 300 mg of nimesulide were prepared as per the procedure of Example 13 but using 300 mg of nimesulide and the tablets were film coated.

### EXAMPLE 18

Controlled release tablets comprising 400 mg of nimesulide were prepared as per the procedure of Example 13, but using 400 mg of nimesulide and the tablets were film coated.

### EXAMPLE 19

A mix of nimesulide (200 mg), mannitol (15 mg) and sodium carboxy methyl cellulose (100 mg) sifted through 20 mesh U.S. screen was granulated using PVP (K-30, 10 mg) dissolved in isopropyl alcohol. The wet mix was sifted through 6 mesh U.S. screen and air dried. The dried granules were sifted through 30 mesh U.S. screen. Methyl cellulose (4000 cps, 50 mg) ethyl cellulose (10 mg), HPMC (K4M, 25 mg) and HPMC (K-100 M, 10 mg) were sifted through 40 mesh U.S. screen and blended with the above granules. The blend was lubricated with talc (10 mg) and magnesium stearate (5 mg) which were previously sifted through 60 mesh U.S. screen. The blend was compressed using suitable punches to obtain floating tablets.

INVIVO STUDIES

Pilot bioequivalence studies using 2×100 mg dosage of a commercially available nimesulide formulation of the immediate release type and 1×200 mg controlled release formulation of Example 1 of the complete specification of the invention were conducted on a single healthy volunteer. The concentrations (C) of nimesulide in plasma at various time intervals were as follows:

TABLE 1

| | | Controlled release |
|---|---|---|
| | Immediate release | formulation of |
| Time | formulation | Example 1 |
| (hrs) | (μg/ml) | (μg/ml) |
| 0 | 0 | 0 |
| 1 | 1.03 | 0.28 |
| 2 | 3.91 | 0.21 |
| 3 | 9.65 | 0.48 |
| 4 | 7.66 | 0.19 |
| 6 | 5.18 | 3.5 |
| 8 | 2.62 | 7.04 |
| 10 | 1.85 | 6.2 |
| 12 | 1.42 | 3.1 |
| 16 | 0.54 | 1.63 |
| 20 | 0.29 | 0.99 |
| 24 | 0.13 | 0.53 |

TABLE 2

| | | | Controlled release | |
|---|---|---|---|---|
| | Immediate release | | formulation of | |
| Parameters | formulation | | Example 1 | |
| Cₘₐₓ | 9.65 | μg/ml | 7.04 | μg/ml |
| Tₘₐₓ | 3.0 | hrs | 8.0 | hrs |
| AUC | 53.22 | units | 55.29 | units |

From the above data it is clear that the controlled release formulation of the invention provided maximum concentration of nimesulide at about 8 hours (Cₘₐₓ=7.04 μg/ml, Tₘₐₓ=8 hours) following administration, as against the maximum concentration achieved as early as within 3 hours with the immediate release formulation. Thus the formulation of the invention provides for sustained effect of nimesulide. Further the plasma concentration of nimesulide at the end of 12 and 20 hours following administration of the formulation of the invention are much higher than those with the immediate release formulation, further eliciting the sustained effect achieved with the formulation of the invention. The results of AUC (Area Under Curve) of the immediate release formulation and the formulation of the invention from Table 2 confirm that the formulation of the invention in addition to providing sustained effect, shows adequate bioequivalency of the order of 103.89%.

1. A controlled release anti-inflammatory nimesulide oral formulation comprising 45-65% by weight of nimesulide and 10-30% by weight of hydrophilic polymer as retardant with pharmaceutically acceptable auxiliary agents and/or excipients; wherein the nimesulide is partially or fully dispersed in the hydrophilic polymer.

2. A formulation as claimed in claim 1, wherein the nimesulide is in 50-55% by weight and the retardant is in 15-20% by weight.

3. A formulation as claimed in claim 1, which is a partially controlled release formulation comprising 25-65% by weight of the nimesulide.

4. A formulation as claimed in claim 1, wherein the hydrophilic polymer is hydroxy propyl methyl cellulose or ethyl cellulose.

5. A formulation as claimed in claim 1, wherein the pharmaceutically acceptable auxiliary agents are diluent, binder, disintegrant or mixtures thereof.

6. A formulation as claimed in claim 1, wherein the pharmaceutically acceptable auxiliary agent is diluent microcrystalline cellulose and/or mannitol in 5-20% by weight.

7. A formulation as claimed in claim 1, wherein the pharmaceutically acceptable auxiliary agent is binder polyvinyl pyrrolidone and/or starch in 2-5% by weight.

8. A formulation as claimed in claim 1, wherein the pharmaceutically acceptable auxiliary agent is disintegrant starch and/or sodium starch glycolate in 2-3 % by weight.

9. A formulation as claimed in claim 1, which is in the form of tablets, bilayered tablets or floating tablets.

10. A formulation as claimed in claim 1, which comprises 100-600 mg of the nimesulide.

11. A process for the preparation of a controlled release anti-inflammatory nimesulide oral formulation comprising mixing 45-65% by weight of nimesulide with 10-30% by weight of hydrophilic polymer as retardant and pharmaceutically acceptable auxiliary agents and/or excipients; wherein the nimesulide is partially or fully dispersed in the hydrophilic polymer.

12. A process as claimed in claim 11, wherein the nimesulide is in 50-55% by weight and the retardant is in 15-20% by weight.

13. A process as claimed in claim 11, wherein the formulation is a partially controlled release formulation comprising 25-65% by weight of the nimesulide.

14. A process as claimed in claim 11, wherein the hydrophilic polymer is hydroxy propyl methyl cellulose or ethyl cellulose.

15. A process as claimed in claim 1, wherein the pharmaceutically acceptable auxiliary agents are diluent, binder disintegrant or mixtures thereof.

16. A process as claimed in claim 11, wherein the pharmaceutically acceptable auxiliary agent is diluent microcrystalline cellulose and/or mannitol in 5-20% by weight.

17. A process as claimed in claim 11, wherein the pharmaceutically acceptable auxiliary agent is binder polyvinyl pyrrolidone and/or starch in 2-5% by weight.

18. A process as claimed in claim 11, wherein the pharmaceutically acceptable auxiliary agent is disintegrant starch and/or sodium starch glycolate in 2-3 % by weight.

19. A process as claimed in claim 11, wherein the formulation is in the form of tablets, bilayered tablets or floating tablets.

20. A process as claimed in claim 11, wherein the nimesulide is in 100-600 mg.
